(19) United States
(12) Patent Application Publication
(10) Pub. No.: US 2013/0261685 A1
(43) Pub. Date: Oct. 3, 2013

(54) COORDINATED HIS-BUNDLE PACING AND HIGH ENERGY THERAPY

(71) Applicant: Cardiac Pacemakers, Inc., St. Paul, MN (US)

(72) Inventors: Allan C. Shuros, St. Paul, MN (US); Jiang Ding, Shoreview, MN (US); Dan Li, Shoreview, MN (US); Yinghong Yu, Shoreview, MN (US)

(21) Appl. No.: 13/852,199

(22) Filed: Mar. 28, 2013

Related U.S. Application Data
(60) Provisional application No. 61/617,297, filed on Mar. 29, 2012.

(51) Int. Cl. A61N 1/39

(52) U.S. Cl. CPC A61N 1/3962
USPC 607/4; 607/5

(57) ABSTRACT

A cardiac arrhythmia can be identified, such as a tachycardia or fibrillation episode (atrial or ventricular). In responses to the detected arrhythmia, a coordinated electrostimulation therapy can be provided using at least one of a defibrillation shock therapy, a pre-shock conditioning therapy, or a post-shock conditioning therapy. The pre-shock or post-shock conditioning therapies can include electrostimulation therapies provided to the natural electrical conduction system of the heart between the atrioventricular node and the Purkinje fibers, inclusive, such as at or near a His bundle of a heart. In an example, a defibrillation threshold can be reduced by providing a pre-shock conditioning electrostimulation therapy to the natural electrical conduction system of the heart between the atrioventricular node and the Purkinje fibers, inclusive, such as at or near a His bundle.

### CLAIM OF PRIORITY

This application claims the benefit of priority under 35 U.S.C. §119(e) of Shuros et al., U.S. Provisional Patent Application Ser. No. 61/617,297, entitled “COORDINATED HIS-BUNDLE PACING AND HIGH ENERGY THERAPY”, filed on Mar. 29, 2012, which is incorporated herein by reference in its entirety.

### BACKGROUND

A natural cardiac activation sequence can include an electrical impulse that can originate at a sinoatrial node (SA node), pass through intermodal atrial pathways, and arrive at an atrioventricular node (AV node). From the AV node, a His bundle and its various branches can be activated, and electrical signals can ultimately reach an apex of the myocardium using a heart's Purkinje system. In some patients, the natural cardiac activation sequence can be disturbed. For example, abnormal activation of myocytes, or individual cardiac muscle cells or groups of muscle cells, can cause cardiac arrhythmia episodes.

Intrinsic electrical stimuli of the heart, such as in the presence of various myocardial substrate modifications (e.g., infarcted, non-conducting, or reduced-conduction areas), can re-enter an original activation circuit and trigger a new activation. Such re-entrant circuits can lead to elevated heart rates that can be fatal. For example, tachycardia or fibrillation episodes can occur.

Medical devices, such as implantable medical devices, can be used to perform one or more tasks including monitoring, detecting, or sensing physiological information in or otherwise associated with the body, diagnosing a physiological condition or disease, treating or providing a therapy for a physiological condition or disease, or restoring or otherwise altering the function of an organ or a tissue. Examples of an implantable medical device can include a cardiac rhythm management device, such as a pacemaker, a cardiac resynchronization therapy device, a cardioverter or defibrillator, a neurological stimulator, a neuromuscular stimulator, or a drug delivery system.

In an example, cardiac rhythm or function management devices can sense heart contractions or deliver electrostimulation to evoke responsive heart contractions. In an example, one or more of these functions can help improve a patient's heart rhythm or can help coordinate a spatial nature of a heart contraction, either of which can improve cardiac output of blood to help meet a patient's metabolic need.

Some cardiac rhythm or function management devices can be configured to deliver energy at or near the His bundle to achieve pacing via natural conduction pathways, such as via Purkinje fiber conduction of electrical impulses. Various devices for delivering signals to an electrode near a His bundle have been proposed. For example, Zhu et al., PCT Patent Publication No. WO 2010/071849, entitled DEVICES, METHODS, AND SYSTEMS INCLUDING CARDIAC PACING, refers to delivering an anti-tachyarrhythmia pacing pulse to an electrode near a His bundle in a right ventricle of a heart.

### OVERVIEW

A cardiac arrhythmia can be identified, such as a tachycardia or fibrillation episode (atrial or ventricular). In response to the detected arrhythmia, a coordinated electrostimulation therapy can be provided using at least one of a defibrillation shock therapy, a pre-shock conditioning therapy, or a post-shock conditioning therapy. The pre-shock or post-shock conditioning therapies can include electrostimulation therapies provided to a natural electrical conduction system of the heart between the AV node and the Purkinje fibers, inclusive, such as at or near a His bundle of a heart. In an example, a defibrillation threshold can be reduced by providing a pre-shock conditioning electrostimulation therapy to the natural electrical conduction system of the heart between the AV node and the Purkinje fibers, inclusive, such as at or near a His bundle.

The present inventors have recognized that, among other things, a problem to be solved can include improving a patient response to a defibrillation shock. In an example, the present subject matter can provide a solution to this problem by reducing a defibrillation threshold, or by promoting post-shock resynchronization of the patient's natural cardiac conduction system using a natural electrical conduction system of the heart. For example, His bundle electrostimulation (e.g., His bundle pacing) can be provided before or after a defibrillation shock.

This overview is intended to provide an overview of subject matter of the present patent application. It is not intended to provide an exclusive or exhaustive explanation of the invention. The detailed description is included to provide further information about the present patent application.

### BRIEF DESCRIPTION OF THE DRAWINGS

In the drawings, which are not necessarily drawn to scale, like numerals may describe similar components in different views. Like numerals having different letter suffixes may represent different instances of similar components. The drawings illustrate generally, by way of example, but not by way of limitation, various embodiments discussed in the present document.

FIG. 1 illustrates generally an example that can include an ambulatory medical device coupled to an external module.

FIG. 2 illustrates generally an example of a system that can be configured to provide a defibrillation therapy.

FIG. 3 illustrates generally an example of a system that can include an IMD coupled to electrode leads disposed in or near a heart.

FIG. 4 illustrates generally an example of an atrial lead.

FIG. 5 illustrates generally an example that can include providing a conditioning electrostimulation or a defibrillation shock.

FIGS. 6A, 6B, and 6C illustrate generally examples of several EKG waveforms.

FIG. 7 illustrates generally an example of a pre-shock conditioning therapy.

FIG. 8 illustrates generally an example of a post-shock conditioning therapy.

FIG. 9 illustrates generally an example of a post-shock conditioning therapy.

FIG. 10 illustrates generally an example of a defibrillation therapy that includes a conditioning therapy.

FIG. 11 illustrates generally an example that can include updating a defibrillation therapy parameter.

FIG. 12 illustrates generally an example that can include a therapy for a persistent arrhythmia episode.

FIG. 13 illustrates generally an example that can include updating a therapy parameter.

FIG. 14 illustrates generally an example of defibrillation threshold experiment data.

### DETAILED DESCRIPTION

A tachycardia or fibrillation episode can be treated by providing a therapy to address an underlying mechanism responsible for maintaining the episode. Fibrillation episodes can include atrial or ventricular fibrillation episodes, and, depending on the duration of a particular fibrillation episode, can be maintained by different physiological mechanisms or responded to using different therapies.

Dosdall et al., in “Purkinje Activation Precedes Myocardial Activation Following Defibrillation after Long-Duration Ventricular Fibrillation,” Heart Rhythm, Vol. 7, No. 3, March 2010, describes identifying post-defibrillation shock activations of the ventricular myocardium (VM) and the Purkinje fibers in dogs. Dosdall et al. observed that, after long-duration ventricular fibrillation (e.g., a fibrillation episode lasting more than one minute), post-defibrillation shock Purkinje fiber activation occurred before VM activation.

The present inventors have recognized that, among other things, a patient response to a defibrillation shock can be improved by promoting resynchronization of the patient's natural cardiac conduction system, such as including the Purkinje system. For example, a defibrillation therapy can be provided by coordinating a defibrillation shock with electrostimulation of a natural electrical conduction system of a heart, such as an electrostimulation provided between the atrioventricular node (AV node) and the Purkinje fibers, inclusive. Such therapies can improve a cardiac substrate response to a defibrillation shock. In an example, a coordinated defibrillation therapy can include at least one of a pre-defibrillation shock conditioning electrostimulation (pre-shock) or a post-defibrillation shock conditioning electrostimulation (post-shock) provided to a location at or near a His bundle.

The present inventors have also recognized, among other things, that arrhythmias can continue, or redevelop, after a defibrillation shock is provided, such as due to re-entrant, or ectopic myocardial activations. A solution to this problem can include preventing continuing or redeveloping arrhythmic activity after a defibrillation shock by promoting a natural cardiac activation sequence, such as using electrostimulation to activate the Purkinje system or the ventricular myocardium.

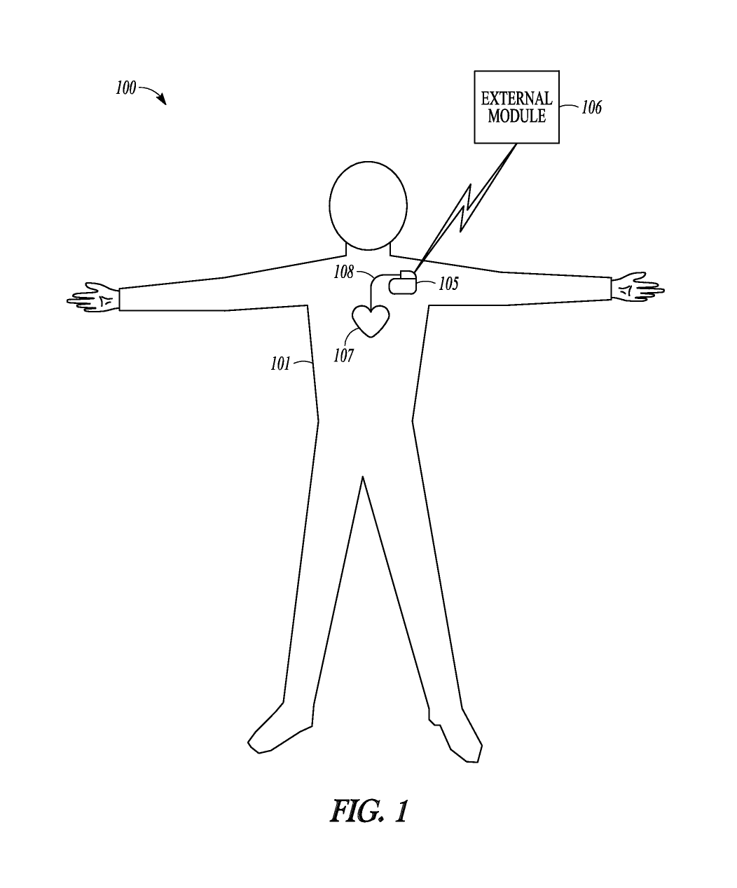

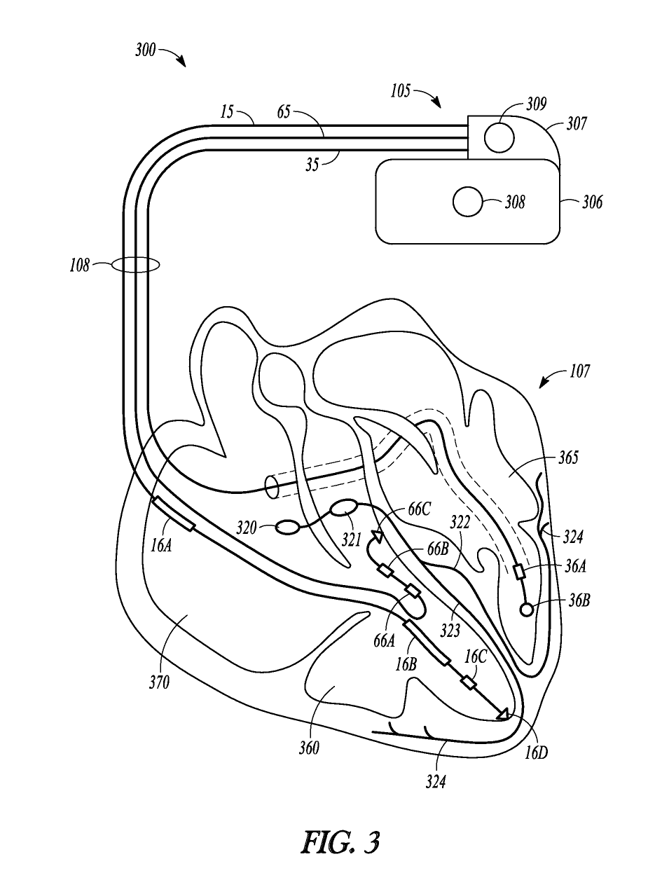

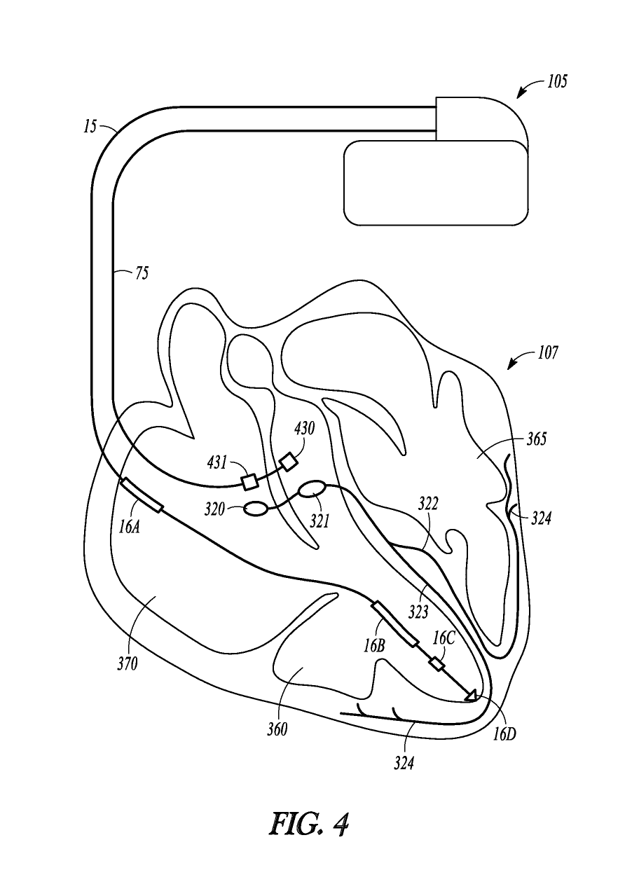

FIG. 1 illustrates generally an example of a system 100 that can provide a coordinated defibrillation therapy. The system 100 can include an ambulatory or implantable medical device (IMD) 105 in a subject 101. The IMD 105 can include a pacemaker, a defibrillator, or one or more other implantable medical devices. In an example, the IMD 105 can be coupled to a lead system 108. The lead system 108 can be coupled to a patient heart 107, and configured to deliver an electrostimulation therapy to the heart 107. Some examples of the IMD 105 and the lead system 108 are discussed below at FIGS. 3 and 4.

The IMD 105 can be coupled, such as wirelessly, to an external module 106. The IMD 105 can include, among other functional portions, one or more of an electrical energy delivery circuit, a detector circuit, or a processor circuit. A portion of the functionality of one or more of the electrical energy delivery circuit, the detector circuit, or the processor circuit, can occur in the IMD 105, and another portion elsewhere (e.g., in an external component, such as a 12-lead EKG detector).

The IMD 105 can include an antenna, such as can be configured to provide radio-frequency or other communication between the IMD 105 and the external module 106, or other external device. The external module 106 can include a local medical device programmer or other local external module, such as within wireless communication range of the IMD 105 antenna.

The external module 106 can include a remote medical device programmer or one or more other remote external modules (e.g., outside of wireless communication range of the IMD 105 antenna, but coupled to the IMD 105, such as using a local external device, such as a repeater or network access point). The external module 106 can be configured to send information to or receive information from the IMD 105. The information can include medical device programming information, such as coordinated defibrillation therapy programming information, subject data, device data, or other instructions, alerts, or other information. The external module 106 can be configured to provide information (e.g., information received from the IMD 105) to a user.

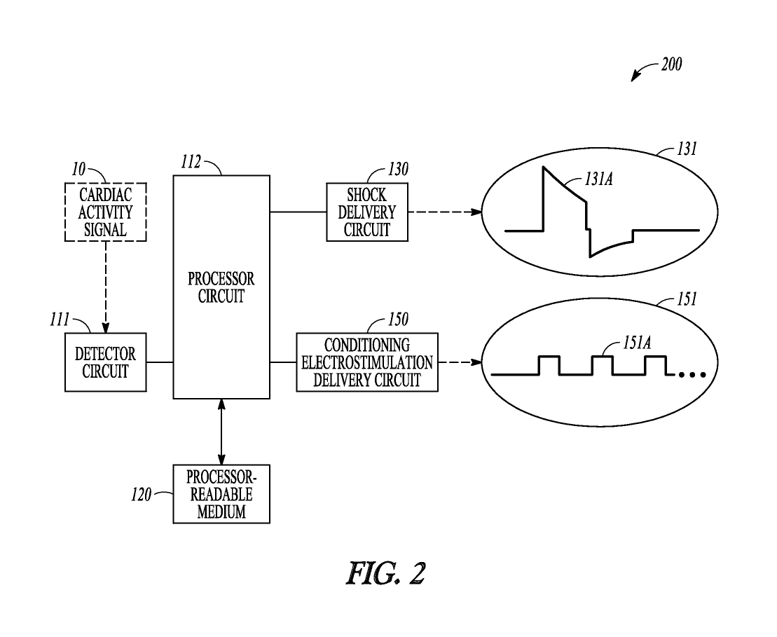

FIG. 2 illustrates generally an example of a system 200 that can be configured to provide a defibrillation therapy. The system 200 can include a detector circuit 111, a processor circuit 112, a processor-readable medium 120, a shock delivery circuit 130, or a conditioning electrostimulation delivery circuit 150. In an example, the delivery circuits 130 and 150 can be configured to generate or provide an electrostimulation, such as can be delivered to a subject body using an implantable or external lead to evoke a cardiac response, such as a cardiac depolarization or contraction.

The shock delivery circuit 130 can be configured to provide a defibrillation shock 131 to a heart. The shock delivery circuit 130 can be coupled to an electrode (e.g., using the lead system 108) configured to provide the defibrillation shock 131 to the heart. The electrode can be a coil electrode disposed in a right ventricle, or a can electrode (e.g., IMD 105 housing electrode), among others. The shock delivery circuit 130 can be configured to provide, among others signals, a defibrillation shock comprising a biphasic waveform 131a.

The conditioning electrostimulation delivery circuit 150 can be configured to provide a conditioning electrostimulation therapy 151, such as in conjunction with the defibrillation shock. The conditioning electrostimulation delivery circuit 150 can be coupled to an electrode configured to stimulate a natural electrical conduction system of the heart between the AV node and the Purkinje fibers, inclusive. In an example, the electrode can be disposed at or near the AV node. In an example, the electrode can be disposed in a right ventricle, such as at one or more locations along an interventricular septum, a right ventricular outflow tract septum, at a right atrium, or at one or more other locations near the His bundle. In an example, the electrode can be disposed at or near the left and/or right bundle branches of the heart. The electrode can be disposed at or near the anterior, posterior, or medial fascicles along the left branch of the heart. In an example, the electrode can be disposed at or near the Purkinje fibers of the heart.

The conditioning electrostimulation delivery circuit 150 can be configured to provide the conditioning electrostimulation therapy 151 using at least partially overlapping first and second electrostimulation signal components, such as in opposite polarity from each other with respect to a reference component. Other conditioning electrostimulation therapies can be used as well, such as comprising non-overlapping biphasic waveforms, or monophasic waveforms 151a. The conditioning electrostimulation therapy 151 can include a series of electrostimulation therapy pulses. Throughout this document, a conditioning electrostimulation can refer to a discrete electrostimulation pulse, or to a series of electrostimulation pulses. For example, a first conditioning electrostimulation can comprise a first series of electrostimulation pulses, and a second conditioning electrostimulation can comprise a second series of electrostimulation pulses.

The detector circuit 111 can be configured to receive a cardiac activity signal 10 (e.g., a signal representative of electrical activity of a heart), for example, over at least a portion of a cardiac cycle. The detector circuit 111 can be configured to receive information such as including one or more of: electrogram or electrical cardiogram (EKG) information (e.g., an evoked response EKG, a subcutaneous EKG, or other electrical activity information); heart sound information, such as can be received from a heart sound sensor such as a microphone; acceleration information, such as can be received from an accelerometer configured to provide an indication of mechanical cardiac activity; pressure information, such as can be received from a pressure sensor configured to provide an indication of a pressure, such as a central venous pressure (CVP); thoracic or other impedance information; or other information indicative of cardiac activity.

The processor circuit 112 can be coupled to the detector circuit 111 and the delivery circuits 130 and 150. The processor circuit 112 can be configured to determine a characteristic of the received cardiac activity signal 10, such as over at least a portion of a cardiac cycle. In an example, a characteristic can include, among others, at least one of a width, amplitude, polarity, slope, or latency of a QRS complex, an R-wave timing, a pressure, an indication of mechanical motion provided by an accelerometer, or an impedance. One or more other characteristics can be used, such as a measure of contractility, synchrony, or cardiac output, among others. The processor circuit 112 can use information from the determined characteristic(s) to identify a cardiac arrhythmia or a fibrillation episode. In response to identifying the cardiac arrhythmia or the fibrillation episode, the processor circuit 112 can initiate an electrostimulation, such as using one or more of the shock delivery circuit 130 or the conditioning electrostimulation delivery circuit 150. For example, in response to a ventricular fibrillation episode, the processor circuit 112 can initiate a pre-shock conditioning therapy using the conditioning electrostimulation delivery circuit 150. The pre-shock conditioning therapy can be provided to a para-Hisian region of a patient's heart.

In an example, the IMD 105 (see, e.g., FIG. 1) can include all or a portion of the system 200. For example, a single implantable medical device can include the detector circuit 111, the processor circuit 112, the processor-readable medium, the shock delivery circuit 130, and the conditioning electrostimulation delivery circuit 150. The components of the system 200 can be distributed among one or more implantable or external medical devices. For example, a first implantable medical device can include the conditioning electrostimulation delivery circuit 150, and a second medical device can include the shock delivery circuit 130. Each of the delivery circuits 130 and 150 can be coupled to a corresponding processor circuit, or can be coupled to a common processor circuit, such as via the external module 106.

FIG. 3 illustrates generally an example of a system 300 that includes the IMD 105 and the lead system 108. The lead system 108 can include a right ventricular apex lead 15, a left ventricular lead 35, and a right ventricular septum lead 65. The lead system 108 can be coupled to the shock delivery circuit 130 or the conditioning electrostimulation delivery circuit 150. The IMD 105 can include a housing 306 (or “can”) and a header 307. In an example, at least a portion of the exterior of the housing 306 or the header 307 can include a housing or “can” electrode 308.

The right ventricular apex lead 15 can include a first electrode 16A that can be configured to be located in the superior vena cava of the heart 107, and a second electrode 16B, a third electrode 16C, and a fourth electrode 16D configured to be located in the right ventricle 360 of the heart 102. The first electrode 16A can include a proximal defibrillation coil electrode, or the second electrode 16B can include a distal defibrillation coil electrode, such as can be configured to deliver a high energy shock (e.g., 0.1 Joule or greater) to the heart 107.

The left ventricular lead 35 can include a fifth electrode 36A or a sensor 36B configured to be located in, on, or near the left ventricle 365 of the heart 102, such as within the coronary vasculature. The sensor 36B can include a distal pacing or sensing electrode, or a pressure sensor. The right ventricular septum lead 65 can include a sixth electrode 66A, a seventh electrode 66B, and an eighth electrode 66C, such as configured to be located along the septum in the right ventricle 360 of the heart 102. The right ventricular septum lead 65 can be configured to provide conditioning electrostimulation therapy along the septum wall, such as to an AV node 320, to a His bundle 321, and/or to left or right bundle branches 322 and 323.

The can electrode 308 can be electrically coupled to at least one other electrode (e.g., the first electrode 16A), or the can electrode 308 can be electrically isolated from other electrodes and capable of independent control. Any of the first electrode 16A through the eighth electrode 66C can include at least one of a coil-type electrode, a ring-type electrode, or a tip electrode.

The right ventricular apex lead 15 can be configured to electrically couple the IMD 105 to at least one of the right ventricle 360, the right atrium 370, or the superior vena cava using at least one electrode (e.g., the first electrode 16A, the second electrode 16B, the third electrode 16C, or the fourth electrode 16D), the left ventricular lead 35 can be configured to electrically couple the IMD 105 to the left ventricle 365 using at least one electrode (e.g., the fifth electrode 36A or the sensor 36B), or the right ventricular septum lead 65 can be configured to electrically couple the IMD 105 to the interventricular septum using at least one electrode (e.g., the sixth electrode 66A, the seventh electrode 66B, or the eighth electrode 66C). At least one of the second electrode 16B, the third electrode 16C, or the fourth electrode 16D, can be configured to be located in, on, or near a right apical region of the heart 102.

FIG. 3 illustrates generally an example of several natural electrical conduction systems of the heart 107. For example, the AV node 320 can be coupled to the His bundle 321. The His bundle 321 can be coupled to the left branch bundle 322 and the right branch bundle 323. The left branch bundle 322 can lead to anterior, posterior, and medial fascicles. The left and right branch bundles 322 and 323 can lead to Purkinje fibers 324 near an apex of the heart 107, such as on the left and right sides of the heart 107. The eighth electrode 66C can be located at or near the His bundle 321 or the AV node 320.

FIG. 4 illustrates generally an atrial lead 75, coupled to a tip electrode 430 and a ring electrode 431, and the right ventricular apex lead 15. The tip electrode 430 and ring electrode 431 can be disposed in the right atrium, such as at or near the His bundle 321. The tip electrode 430 and the ring electrode 431 can be electrically coupled to the conditioning electrostimulation delivery circuit 150, and can be configured to deliver an electrostimulation therapy to the His bundle 321. The atrial lead 75 can be used as a sensor, such as to provide information about a physical displacement of at least a portion of the atrial lead 75 in the subject 101. For example, the atrial lead 75 can be electrically coupled to at least one of the delivery circuits 130 or 150, and to the detector circuit 111. An electrical energy delivery circuit, such as the conditioning electrostimulation delivery circuit 150, can be configured to provide a first signal to the atrial lead 75. The detector circuit 111 can be configured to receive and interpret a second signal in response to the first signal, and the second signal can be indicative of a physical displacement of the atrial lead 75, such as described by Ingle, in U.S. Patent Publication No. 2011/0319772 entitled LEAD MOTION SENSING VIA CABLE MICROPHONICS, which is hereby incorporated by reference in its entirety. A system can thus be deployed to use a single lead to provide electrostimulation to the heart 107, such as to a natural electrical conduction system of the heart 107 between the AV node and the Purkinje fibers, inclusive, and to provide a cardiac diagnostic indication, such as an indication of mechanical cardiac activity.

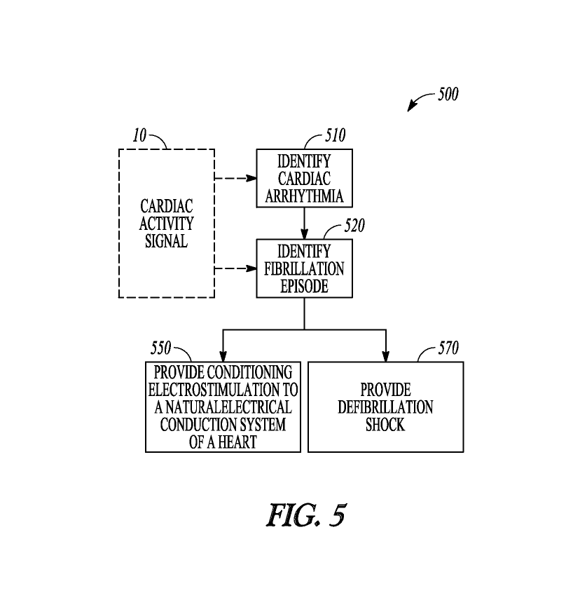

The systems described in FIGS. 1 through 4, among other systems that can be configured to provide or deliver electrostimulation to a patient body, can be used to identify a cardiac arrhythmia or provide a responsive therapy, such as including a defibrillation shock therapy or a conditioning electrostimulation therapy. FIG. 5 illustrates generally an example 500 that can include providing a defibrillation therapy by coordinating a defibrillation shock with a conditioning electrostimulation. In an example, a conditioning electrostimulation can be provided to a natural electrical conduction system of the heart between the AV node and the Purkinje fibers, inclusive, such as at or near a His bundle of a heart.

At 510, a cardiac arrhythmia can be identified. For example, the IMD 105 can receive the cardiac activity signal 10 via the detector circuit 111, and can identify a cardiac arrhythmia using the processor circuit 112, such as using instructions provided via the processor-readable medium 120. Identifying a cardiac arrhythmia can include identifying a heart rate that is too fast, too slow, or irregular. At 520, a fibrillation episode can be identified, such as using the cardiac activity signal 10. For example, when a cardiac arrhythmia is identified at 510, a fibrillation episode, such as a ventricular fibrillation episode, can be distinguished from other arrhythmic activity. At 520, a particular type of fibrillation episode, such as a ventricular fibrillation episode, can be distinguished from an arrhythmia.

The processor circuit 112 can use information about the cardiac activity signal 10 to identify a ventricular fibrillation episode that is contraindicated for an anti-tachycardia pacing therapy. For example, when a tachycardia episode persists for an extended period of time or progresses toward fibrillation, an anti-tachycardia pacing therapy can be contraindicated and a coordinated defibrillation therapy can be provided.

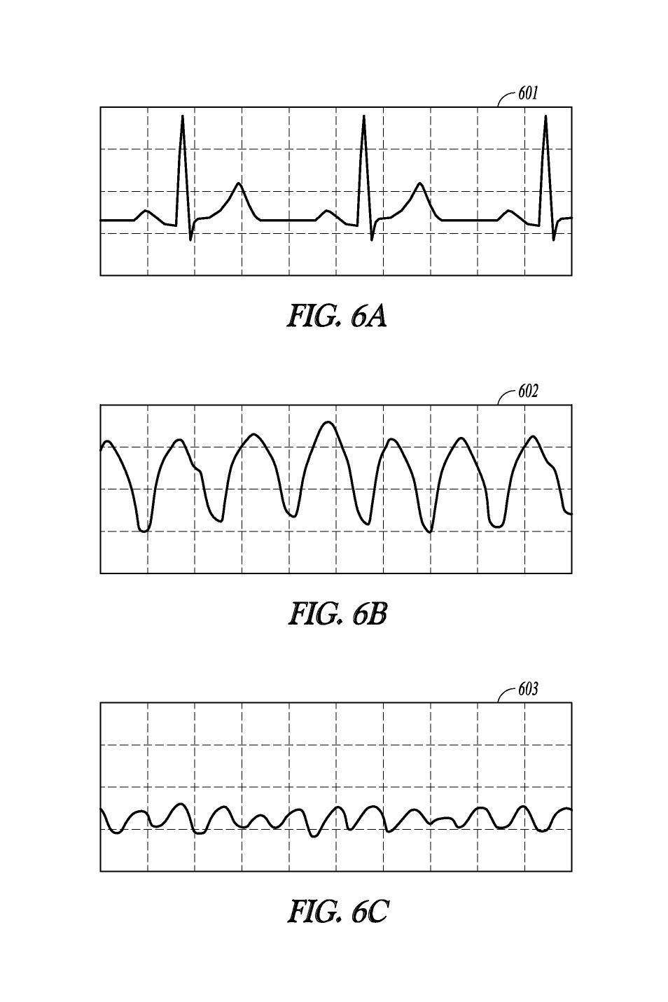

In FIGS. 6A, 6B, and 6C, several examples of EKG waveforms (e.g., from lead II of a 12-lead EKG) illustrate several differences between cardiac activity signals. For example, FIG. 6A illustrates an example of a normal sinus rhythm waveform 601, FIG. 6B illustrates an example of a tachycardia waveform 602, and FIG. 6C illustrates an example of a fibrillation waveform 603. The cardiac activity signal 10 can include EKG information, such as including waveform information, and the processor circuit 112 can use the waveform information to identify a cardiac arrhythmia (e.g., at 510 in the example of FIG. 5) or a fibrillation episode (e.g., at 520 in the example of FIG. 5).

The processor circuit 112 can use a peak-detection algorithm to determine a period of the cardiac activity signal 10 waveform. A first period can be associated with detected peaks of the normal sinus rhythm waveform 601, a second period can be associated with detected peaks of the tachycardia waveform 602, and a third period can be associated with detected peaks of the fibrillation waveform 603. In the example of FIG. 5, at 510, the processor circuit 112 can identify a cardiac arrhythmia when the cardiac activity signal 10 waveform includes the tachycardia waveform 602 having the second period. At 520, the processor circuit 112 can identify a fibrillation episode, such as when the cardiac activity signal 10 waveform includes the fibrillation waveform 603 having the third period.

Referring again to FIG. 5, at 550, a conditioning electrostimulation can be provided, in response to an identified fibrillation episode, to a natural electrical conduction system of the heart between the AV node and the Purkinje fibers, inclusive, such as at or near a His bundle. The conditioning electrostimulation can be provided using the conditioning electrostimulation delivery circuit 150.

The conditioning electrostimulation provided at 550 can be a pre-shock conditioning electrostimulation provided to the natural electrical conduction system before a defibrillation shock. The conditioning electrostimulation provided at 550 can be a post-shock conditioning electrostimulation provided to the natural electrical conduction system after a defibrillation shock. At 570, a defibrillation shock can be provided to a heart, such as before, during, or after the conditioning electrostimulation provided at 550.

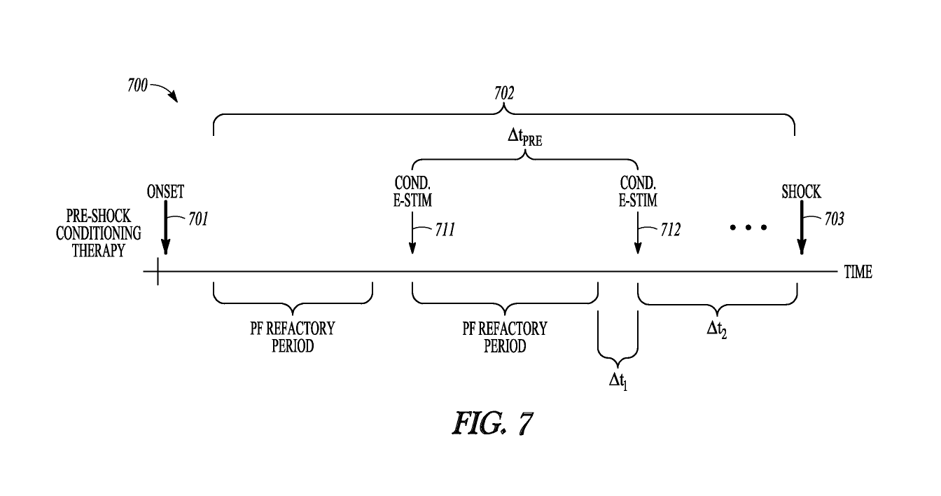

FIG. 7 illustrates generally an example 700 that can include a pre-shock conditioning therapy 702. An onset 701 of a fibrillation episode can be identified (e.g., at 520 in the example of FIG. 5). After a specified duration (e.g., ≧0 ms) from the identified onset 701 of the fibrillation episode, the pre-shock conditioning therapy 702 can be provided, such as using the conditioning electrostimulation delivery circuit 150. The shock delivery circuit 130 can electrically charge a defibrillation component, such as in response to the identified onset 701 of the fibrillation episode, or during the pre-shock conditioning therapy 702.

The pre-shock conditioning therapy 702 can include one or more conditioning electrostimulations provided to the natural electrical conduction system of the heart, such as using the conditioning electrostimulation delivery circuit 150. The pre-shock conditioning therapy 702 can include, as the one or more conditioning electrostimulations, one or more discrete electrostimulation pulses, or one or more series of electrostimulation pulses. In the example of FIG. 7, the pre-shock conditioning therapy 702 can include at least two conditioning electrostimulations, including a first conditioning electrostimulation 711 and a second conditioning electrostimulation 712. The first and second conditioning electrostimulations 711 and 712 can be the same type of signal, or they can be different signals (e.g., the electrostimulations can have the same or different durations, amplitudes, waveform shapes, number of pulses, etc.). For example, the first conditioning electrostimulation 711 can be an electrostimulation having a biphasic waveform shape having first amplitude and duration characteristics, and the second conditioning electrostimulation 712 can be a series of two or more electrostimulations having a biphasic waveform and having second amplitude and duration characteristics.

In an example, an interval Δt_{PRE} can be provided between the first and second conditioning electrostimulations 711 and 712. The interval Δt_{PRE} can be adjustable. The interval Δt_{PRE} can exceed a refractory period of the Purkinje fibers of the heart 107. For example, Δt_{PRE} can exceed a refractory period of the Purkinje fibers by Δt₁, where Δt₁≧0 ms. When the interval Δt_{PRE} exceeds the refractory period of the Purkinje fibers, the pre-shock conditioning therapy 702 can condition or capture the Purkinje system before a defibrillation shock is administered.

In an example, the interval Δt_{PRE} can be less than a refractory period of the Purkinje fibers of the heart 107. When the interval Δt_{PRE} is less than the refractory period of the Purkinje fibers, at least a portion of the Purkinje system can be maintained in a refractory state. By maintaining the Purkinje system in a refractory state, the system can be prevented from unwanted excitation, such as due to retrograde conduction of an arrhythmia.

The pre-shock conditioning therapy 702 can be synchronized with a defibrillation shock 703. For example, the pre-shock conditioning therapy 702 can include an adjustable interval Δt₂ (e.g., Δt₂≧0 ms) between a final conditioning electrostimulation (e.g., the second conditioning electrostimulation 712) and the defibrillation shock 703.

FIGS. 8-10 illustrate generally examples that can include a post-shock conditioning electrostimulation therapy. A post-shock conditioning electrostimulation therapy can be provided to the natural electrical conduction system of the heart, such as to activate the Purkinje system, such that initial, post-shock cardiac activity can occur via a heart's natural conduction system. A post-shock ectopic beat can originate in the ventricular myocardium and re-start an arrhythmia. By activating the Purkinje system using a post-shock conditioning therapy, such ectopic activity can be reduced or eliminated. In an example, after a defibrillation shock therapy, there can be an extended period of time with little or no cardiac activity. This period of cardiac inactivity can be hemodynamically harmful to a patient. For example, the patient's metabolic need for blood throughout the body can be unfulfilled, or the heart can fill with blood, which can stretch the myocardium or cause another arrhythmic episode.

In an example, a post-shock conditioning therapy can include a series of electrostimulations provided to the natural electrical conduction system of the heart between the AV node and the Purkinje fibers, inclusive. The series of electrostimulations can include gradually increasing intervals, such as between adjacent electrostimulations, to allow intrinsic cardiac function to return. The electrostimulations can be provided at an initial rate of about 100 electrostimulations per minute. In an example, the electrostimulations can be provided at an initial rate that is less than or greater than 100 electrostimulations per minute. The rate can be gradually decreased, such as to about 50 electrostimulations per minute, or until intrinsic cardiac function resumes. In an example, the post-shock conditioning therapy can include the series of electrostimulations provided to a location at or near the His bundle.

FIG. 8 illustrates generally an example 800 that can include a fixed-interval post-shock conditioning therapy 802. In an example, the defibrillation shock 703 can be identified. After a specified duration (e.g., ≧0 ms) from the identified shock, the fixed-interval post-shock conditioning therapy 802 can be provided using the conditioning electrostimulation delivery circuit 150. The shock delivery circuit 130 can electrically charge a defibrillation component while the fixed-interval post-shock conditioning therapy 802 is provided, such as to provide a subsequent shock.

The fixed-interval post-shock conditioning therapy 802 can include one or more conditioning electrostimulations, such as using the conditioning electrostimulation delivery circuit 150. The fixed-interval post-shock conditioning therapy 802 can include, as the one or more conditioning electrostimulations, n discrete electrostimulation pulses, or n series of electrostimulation pulses. In the example of FIG. 8, the fixed-interval post-shock conditioning therapy 802 can include at least four conditioning electrostimulations (e.g., n=4), including first, second, third, and fourth conditioning electrostimulations 811, 812, 813, and 814. Any of the post-shock conditioning electrostimulations can be the same type of signal, or they can be different signals (e.g., the electrostimulations can have the same or different durations, amplitudes, waveform shapes, number of pulses, etc.).

An initial post-shock conditioning electrostimulation can be synchronized with the defibrillation shock 703. For example, the fixed-interval post-shock conditioning therapy 802 can include an adjustable interval Δt_{POST-i} (e.g., Δt_{POST-i}≧0 ms) between the defibrillation shock 703 and the first post-shock conditioning electrostimulation 811.

Adjacent conditioning electrostimulations of the fixed-interval post-shock conditioning therapy 802 can be separated by a fixed interval. For example, an interval Δt_{POST} can be provided between each adjacent electrostimulation. In the example of FIG. 8, the first and second conditioning electrostimulations 811 and 812 can be separated by the interval Δt_{POST}, and second and third conditioning electrostimulations 812 and 813 can be separated by the interval Δt_{POST}, and so on. The interval Δt_{POST} can be less than or greater than a refractory period of the Purkinje fibers of the heart 107.

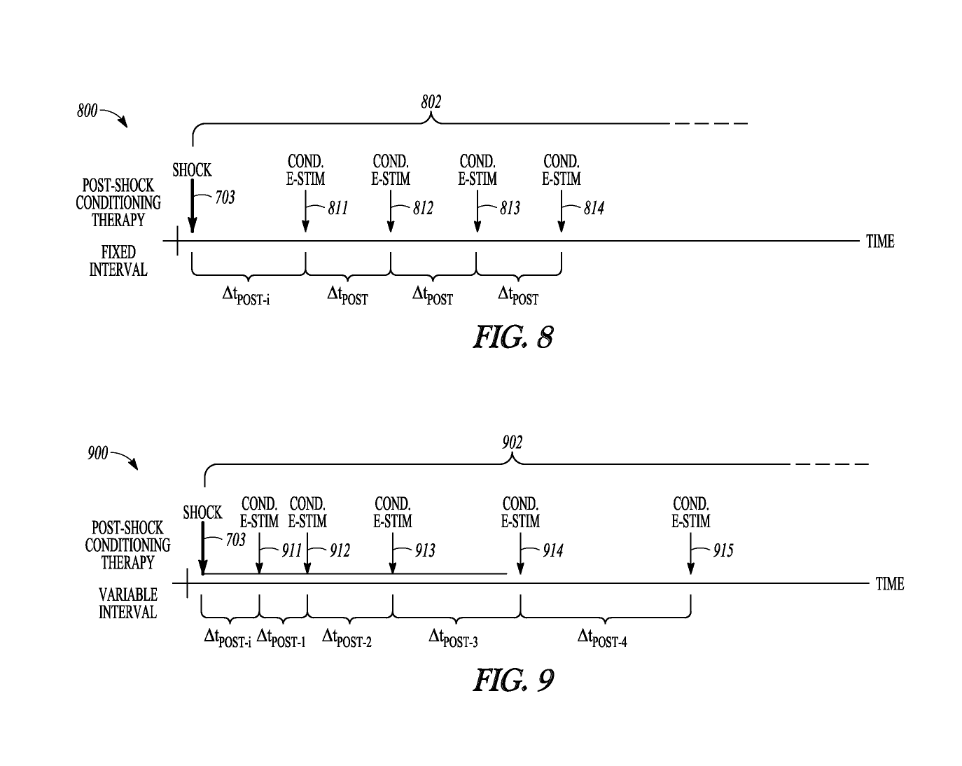

FIG. 9 illustrates generally an example 900 that can include a variable-interval post-shock conditioning therapy 902. The defibrillation shock 703 can be identified. After a specified duration (e.g., ≧0 ms) from the identified shock, the variable-interval post-shock conditioning therapy 902 can be provided, such as using the conditioning electrostimulation delivery circuit 150. The shock delivery circuit 130 can electrically charge a defibrillation component while the variable-interval post-shock conditioning therapy 902 is provided, such as to prepare for subsequent shock delivery.

The variable-interval post-shock conditioning therapy 902 can include one or more conditioning electrostimulations, such as using the conditioning electrostimulation delivery circuit 150. The variable-interval post-shock conditioning therapy 902 can include, as the one or more conditioning electrostimulations, n discrete electrostimulation pulses, or n series of electrostimulation pulses. In the example of FIG. 9, the variable-interval post-shock conditioning therapy 902 can include at least five conditioning electrostimulations (e.g., n=5), including a first, second, third, fourth, and fifth conditioning electrostimulation 911, 912, 913, 914, and 915. Any of the post-shock conditioning electrostimulations can be the same type of signal, or they can be different signals (e.g., the electrostimulations can have the same or different durations, amplitudes, waveform shapes, number of pulses, etc.).

An initial post-shock conditioning electrostimulation can be synchronized with the defibrillation shock 703. For example, the variable-interval post-shock conditioning therapy 902 can include an adjustable interval Δt_{POST-i} (e.g., Δt_{POST-i}≧0 ms) between the defibrillation shock 703 and the first post-shock conditioning electrostimulation 911.

Adjacent conditioning electrostimulations of the variable-interval post-shock conditioning therapy 902 can be separated by a fixed or variable interval. In the example of FIG. 9, for example, the first and second conditioning electrostimulations 911 and 912 can be separated by an interval Δt_{POST-1}, the second and third conditioning electrostimulations 912 and 913 can be separated by the interval Δt_{POST-2}, the third and fourth conditioning electrostimulations 913 and 914 can be separated by the interval Δt_{POST-3}, and so on. The intervals Δt_{POST-1}, Δt_{POST-2}, Δt_{POST-3}, etc. can be sequentially increasing in duration (e.g., Δt_{POST-2} can be a longer duration than the interval Δt_{POST-1}, and the interval Δt_{POST-3} can be a longer duration than the interval Δt_{POST-2}, etc.), such as to gradually allow a heart to regain intrinsic function. Any one or more of the intervals Δt_{POST-n} can be the same. Any of the intervals Δt_{POST-i} or Δt_{POST-n} can be less than or greater than a refractory period of the Purkinje fibers of the heart 107.

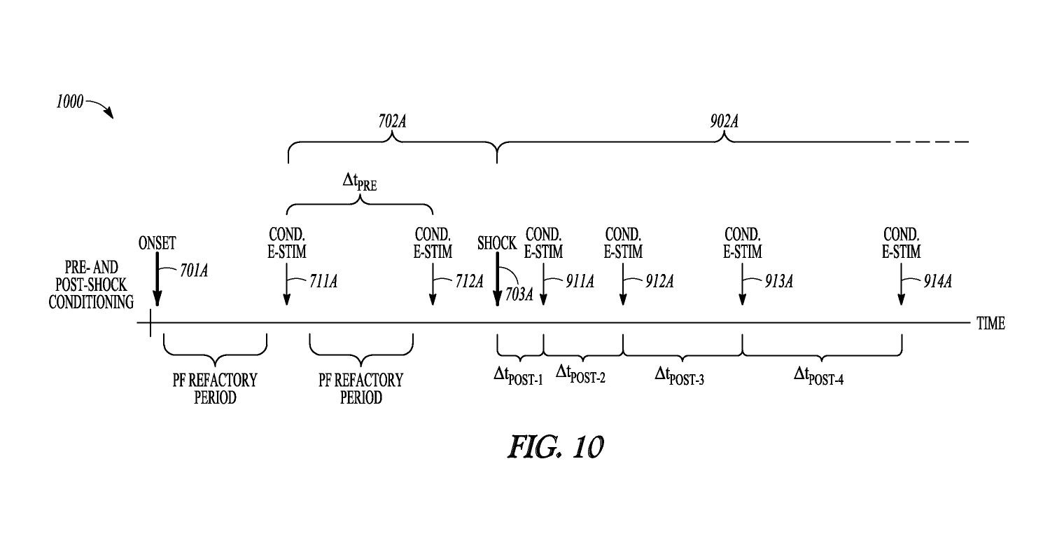

FIG. 10 illustrates generally an example 1000 that can include a defibrillation therapy comprising pre-shock and post-shock conditioning therapies. The example 1000 includes an identified onset 701a of a fibrillation episode. After a specified duration following the identified onset 701a, a pre-shock conditioning therapy 702a can be provided, such as described above in the discussion of FIG. 7 at 702. For example, the pre-shock conditioning therapy 702a can be provided using the conditioning electrostimulation delivery circuit 150, or can be synchronized with the defibrillation shock 703. The pre-shock conditioning therapy 702a can include multiple adjacent pre-shock conditioning electrostimulations 711a, 712a, among others, that can be provided at fixed or variable intervals.

The example 1000 can include a defibrillation shock therapy 703a. After a specified duration following the defibrillation shock therapy 703a, a post-shock conditioning therapy 902a can be provided, such as described above in the discussion of FIGS. 8 and 9. For example, the post-shock conditioning therapy 902a can be provided using the conditioning electrostimulation delivery circuit 150, and can be synchronized with the defibrillation shock therapy 703a. The post-shock conditioning therapy 902a can include multiple adjacent post-shock conditioning electrostimulations, 911a, 912a, 913a, 914a, among others, that can be separated by fixed or variable intervals. The multiple adjacent post-shock conditioning electrostimulations can be provided at increasing intervals (e.g., decreasing in frequency) to allow a heart to gradually regain intrinsic function.

A defibrillation therapy can be provided using a therapy parameter that can determine one or more characteristics of the defibrillation therapy. For example, a defibrillation therapy can include, among other components, a pre-shock conditioning therapy, a defibrillation shock therapy, or a post-shock conditioning therapy (see, e.g., FIG. 10), each of which can be provided using the same or different therapy parameters.

A pre-shock or post-shock conditioning therapy can include one or more conditioning electrostimulations that can be provided to a natural electrical conduction system of the heart, such as at or near a His bundle of a heart. A defibrillation shock therapy can include one or more defibrillation electrostimulations configured to depolarize a heart. The conditioning and defibrillation electrostimulations can be provided using various parameters that can determine characteristics of the electrostimulations. For example, parameters can be used to determine timing or morphology characteristics, such as waveform shape, amplitude, duration, or phase, among other characteristics, of an electrostimulation. A parameter can be used to determine a number of electrostimulations in a therapy, or to determine a number of electrostimulation pulses, such as within a particular electrostimulation event. A parameter can be used to determine an interval between conditioning electrostimulations (e.g., the intervals Δt₁, Δt₂, Δt_{PRE}, Δt_{POST-n}, Δt_{POST-i}, among others.). One or more parameters can be provided to the processor circuit 112, such as via the processor-readable medium 120 or the external module 106. For example, a clinician using an external device programmer (e.g., the external module 106) can update or adjust a conditioning or defibrillation therapy parameter. The processor circuit 112 and/or the delivery circuits 130 and 150 can use the parameter to adjust all or a portion of a defibrillation therapy. A selectable profile comprising one or more parameters (e.g., a set of parameters) can be used to expediently adjust a patient therapy.

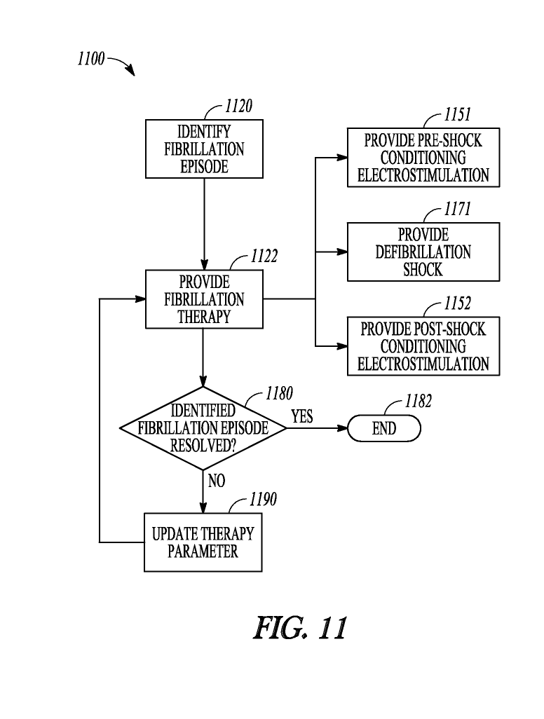

FIG. 11 illustrates generally an example 1100 that can include updating a therapy parameter. At 1120, a fibrillation episode can be identified, such as according to the discussion of FIG. 5 at 520. At 1122, a defibrillation therapy can be provided. The defibrillation therapy can be provided using at least one of the shock delivery circuit 130 or the conditioning electrostimulation delivery circuit 150, such as according to one or more therapy parameters. Providing the defibrillation therapy can include providing one or more of a pre-shock conditioning electrostimulation therapy, a post-shock conditioning electrostimulation therapy, or a defibrillation shock therapy. At 1151, a pre-shock conditioning electrostimulation therapy can be provided at or near a natural electrical conduction system of the heart, such as according to the discussion of FIG. 7. For example, a series of His bundle conditioning electrostimulations can be provided, such as at an interval greater than a refractory period of the Purkinje fibers. At 1171, a defibrillation shock therapy can be provided. The defibrillation shock therapy can be synchronized with a pre-shock conditioning electrostimulation, such as according to the discussion of FIG. 7. At 1152, a post-shock conditioning electrostimulation therapy can be provided, such as according to the discussion of FIG. 8 or 9.

At 1180, cardiac function can be assessed. For example, the detector circuit 111 can be configured to receive post-shock cardiac activity information (e.g., using the cardiac activity signal 10) to determine whether an arrhythmia persists. The processor circuit 112 can determine whether the previously-identified fibrillation episode was resolved. If the fibrillation episode was resolved by the defibrillation therapy, the process can end at 1182. At 1190, if the fibrillation episode (or other arrhythmia) was not resolved by the defibrillation therapy, a therapy parameter can be updated, such as automatically using the processor circuit 112. For example, if an initial defibrillation therapy provided using a first parameter is unsuccessful, a subsequent defibrillation therapy can be provided using a second parameter. For example, the second parameter can be used to provide a defibrillation therapy that is more aggressive than the initial therapy (e.g., the second therapy can include a higher amplitude electrostimulation, such as provided at an increased rate). Updating the therapy parameter at 1190 can include providing the same initial therapy parameter for at least one subsequent defibrillation attempt. After the therapy parameter is updated at 1190, the example 1100 can return to 1122 to provide a subsequent defibrillation therapy, such as using the updated therapy parameter.

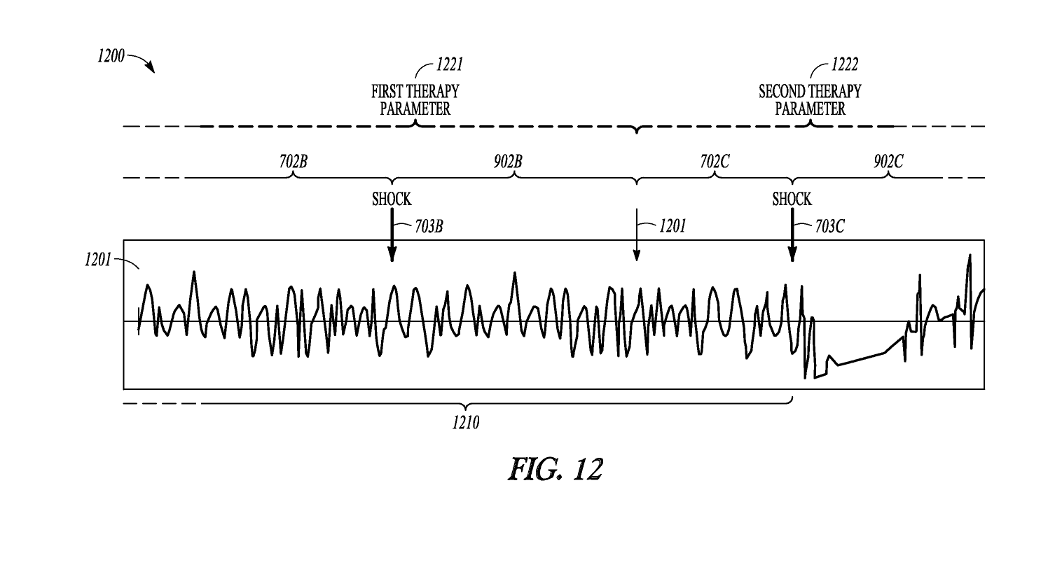

FIG. 12 illustrates generally an example of a persistent fibrillation episode that can be treated using the method described in FIG. 11. In the example of FIG. 12, an EKG waveform 1201 indicates a fibrillation episode 1210. After some initial duration following an onset of the fibrillation episode 1210, a pre-shock conditioning electrostimulation therapy 702b can be provided, such as according to the discussion of FIG. 7 and/or according to the discussion of FIG. 11 at 1151. The pre-shock conditioning electrostimulation therapy 702b can be provided using a first therapy parameter 1221. The first therapy parameter 1221 can include one or more parameters that can be used to determine characteristics of the pre-shock conditioning electrostimulation therapy 702b, among other therapies.

A defibrillation shock therapy 703a can be provided, such as in coordination with the pre-shock conditioning electrostimulation therapy 702b and using the first therapy parameter 1221. The defibrillation shock therapy 703a can be provided according to the discussion of FIG. 11 at 1171. A post-shock conditioning electrostimulation therapy 902b can be provided using the first therapy parameter 1221. The post-shock conditioning electrostimulation therapy 902b can be provided according to the discussion of FIG. 11 at 1152.

At 1201, a persistent arrhythmia episode can be identified, such as according to the discussion of FIG. 11 at 1180. For example, at 1201, the processor circuit 112, such as together with the detector circuit 111, can determine that a fibrillation episode was not successfully treated using the therapies 702b, 703b, and 902b. In response, one or more therapy parameters can be updated, such as according to the discussion of FIG. 11 at 1190. For example, parameters that determine one or more characteristics of the defibrillation shock therapy, the pre-shock conditioning electrostimulation therapy, or the post-shock conditioning electrostimulation therapy can be updated. In an example, the first therapy parameter 1221 can be updated, and a second therapy parameter 1222 can be provided.

After the persistent arrhythmia episode is identified at 1201, a subsequent defibrillation therapy can be provided using the second therapy parameter 1222. The subsequent defibrillation therapy can include, among other therapies, a pre-shock conditioning electrostimulation therapy 702c, a defibrillation shock therapy 703c, or a post-shock conditioning electrostimulation therapy 902c.

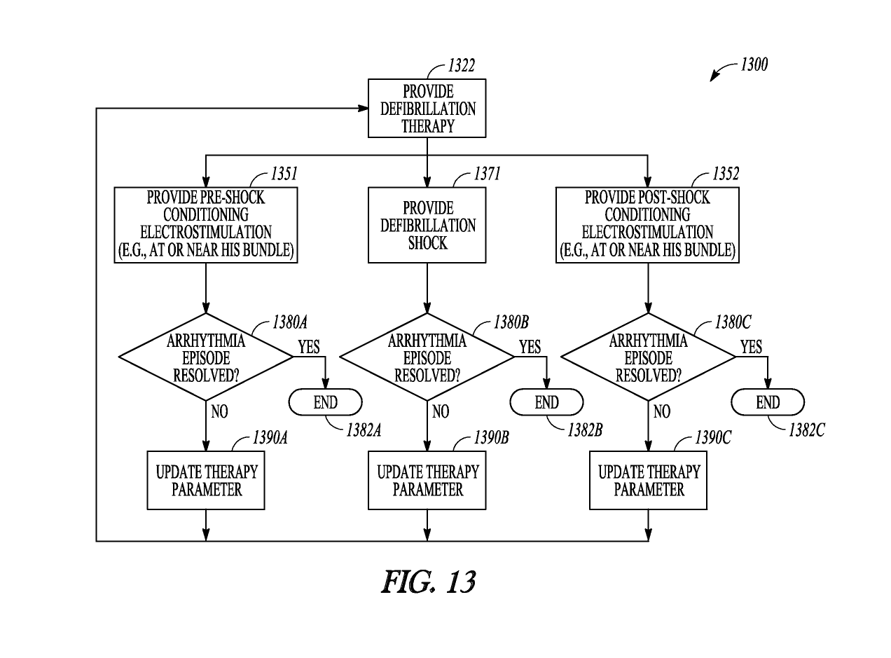

FIG. 13 illustrates generally an example 1300 that can include assessing an arrhythmia episode during a defibrillation therapy. At 1322, a defibrillation therapy can be provided, such as according to the discussion of FIG. 11 at 1122. The defibrillation therapy can include, among other therapies, a pre-shock conditioning electrostimulation therapy, a defibrillation shock therapy, or a post-shock conditioning electrostimulation therapy.

At 1351, a pre-shock conditioning electrostimulation therapy can be provided, such as according to the discussion of FIG. 11 at 1151. At 1380A, such as during or after the pre-shock conditioning electrostimulation therapy, an arrhythmia episode can be assessed. For example, assessing the arrhythmia can include determining whether an arrhythmia persists or whether a characteristic of the arrhythmia has changed, such as in response to the pre-shock conditioning electrostimulation therapy. If the arrhythmia episode is resolved, the process can terminate at 1382A, or it can proceed to another type of therapy. At 1390A, a therapy parameter can be updated, such as when the arrhythmia episode is not resolved. The updated therapy parameter can include a therapy parameter configured for use with an ongoing or subsequent pre-shock conditioning electrostimulation therapy, or for use with one or more other therapies (e.g., a defibrillation shock therapy or a post-shock conditioning electrostimulation therapy). After the therapy parameter is updated at 1390A, the example 1300 can return to 1322, such as to provide another portion of a defibrillation therapy.

At 1371, a defibrillation shock therapy can be provided, such as according to the discussion of FIG. 11 at 1171. At 1380B, such as during or after the defibrillation shock therapy, an arrhythmia episode can be assessed. The assessed arrhythmia can be from an earlier assessed episode, such as the episode assessed at 1380A. Assessing the arrhythmia can include determining whether an arrhythmia persists or whether a characteristic of the arrhythmia has changed, such as in response to the pre-shock conditioning electrostimulation therapy or the defibrillation shock therapy. If the arrhythmia episode is resolved, the process can terminate at 1382B, or it can proceed to another type of therapy. At 1390B, a therapy parameter can be updated, such as when the arrhythmia episode is not resolved. The updated therapy parameter can include a therapy parameter configured for use with an ongoing or subsequent defibrillation shock therapy, or for use with one or more other therapies (e.g., a pre-shock or post-shock conditioning electrostimulation therapy). After the therapy parameter is updated at 1390B, the example 1300 can return to 1322, such as to provide another portion of a defibrillation therapy.

At 1352, a post-shock conditioning electrostimulation therapy can be provided, such as according to the discussion of FIG. 11 at 1152. At 1380C, such as during or after the post-shock conditioning electrostimulation therapy, an arrhythmia episode can be assessed. For example, assessing the arrhythmia can include determining whether an arrhythmia persists or whether a characteristic of the arrhythmia has changed, such as in response to the pre-shock conditioning electrostimulation therapy, the defibrillation shock therapy, or the post-shock conditioning electrostimulation therapy. If the arrhythmia episode is resolved, the process can terminate at 1382C, or it can proceed to another type of therapy. At 1390C, a therapy parameter can be updated, such as when the arrhythmia episode is not resolved. The updated therapy parameter can include a therapy parameter configured for use with an ongoing or subsequent post-shock conditioning electrostimulation therapy, or for use with one or more other therapies (e.g., a defibrillation shock therapy or a pre-shock conditioning electrostimulation therapy). After the therapy parameter is updated at 1390C, the example 1300 can return to 1322, such as to provide another portion of a defibrillation therapy.

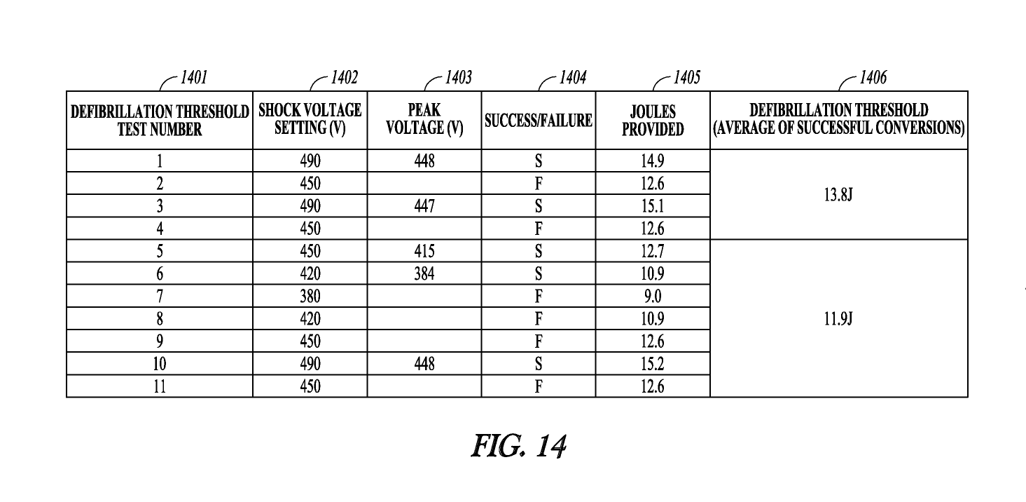

A pre-shock conditioning electrostimulation therapy, such as provided to a natural electrical conduction system of the heart, can be used to reduce a defibrillation threshold. FIG. 14 illustrates an example of actual data from defibrillation threshold tests performed on swine. In the example of FIG. 14, the first column 1401 indicates a defibrillation threshold test number. The second column 1402 indicates a defibrillation shock therapy voltage setting. The third column 1403 indicates the peak defibrillation shock therapy voltage magnitude. The fourth column 1404 indicates whether a defibrillation shock therapy, at the corresponding peak voltage magnitude, successfully converted a fibrillation episode to a normal sinus rhythm. The fifth column 1405 indicates the defibrillation energy provided. The sixth column 1406 indicates an average defibrillation threshold corresponding to the successful conversions. Defibrillation threshold test numbers 1 through 4 were performed without a pre-shock conditioning electrostimulation therapy. The average defibrillation threshold corresponding to successful conversions without a pre-shock conditioning therapy was about 13.8 J. Defibrillation threshold test numbers 5 through 11 were performed with a pre-shock conditioning electrostimulation therapy. The pre-shock conditioning electrostimulation therapy included a biphasic, single-pulse electrostimulation signal with a duration of about 12 to 18 ms. The pre-shock conditioning electrostimulation therapy was applied to the His bundle of the swine heart at a rate of about 150 ms. The average defibrillation threshold corresponding to successful conversions with a pre-shock conditioning therapy was about 11.9 J. Accordingly, a defibrillation threshold can be effectively reduced using a pre-shock conditioning therapy, such as a pre-shock conditioning electrostimulation therapy provided to a His bundle.

### Various Notes & Examples

Example 1 can include subject matter (such as an apparatus, a method, a means for performing acts, or a machine readable medium including instructions that, when performed by the machine, that can cause the machine to perform acts), such as can include or use a detector circuit and an electrical energy delivery circuit. The detector circuit can be configured to receive a signal representative of electrical activity of a heart. Example 1 can include or use a first electrical energy delivery circuit configured to provide a defibrillation shock to be delivered to the heart, and a second electrical energy delivery circuit configured to provide a conditioning electrostimulation, such as in conjunction with the defibrillation shock, to be delivered to a natural electrical conduction system of the heart between the atrioventricular node and the Purkinje fibers, inclusive. For example, the second electrical energy delivery circuit can be configured to provide the conditioning electrostimulation to a location at or near a His bundle of the heart, at or near the left or right bundle branch of the heart, or at or near the anterior, posterior, or medial fascicles along the left branch of the heart. Example 1 can include or use a processor circuit, such as can be coupled to the detector circuit and the first and second electrical energy delivery circuits. In Example 1, the processor circuit configured to identify a fibrillation episode using the received signal representative of electrical activity of the heart, and in response to the identified fibrillation episode, initiate a defibrillation therapy. In Example 1, the defibrillation therapy can include a defibrillation shock, such as provided using the first electrical energy delivery circuit, and a conditioning electrostimulation, such as provided using the second electrical energy delivery circuit.

Example 2 can include, or can optionally be combined with the subject matter of Example 1, to optionally include the processor circuit configured to identify the fibrillation episode, including the processor circuit configured to distinguish the fibrillation episode from a tachycardia episode.

Example 3 can include, or can optionally be combined with the subject matter of one or any combination of Examples 1 or 2 to optionally include the processor circuit configured to identify, as the fibrillation episode, a ventricular fibrillation episode that is contraindicated for an anti-tachycardia pacing therapy.

Example 4 can include, or can optionally be combined with the subject matter of one or any combination of Examples 1 through 3 to optionally include a conditioning electrostimulation that comprises a pre-shock conditioning electrostimulation, provided before the defibrillation shock, using the second electrical energy delivery circuit.

Example 5 can include, or can optionally be combined with the subject matter of one or any combination of Examples 1 through 4 to optionally include the second electrical energy delivery circuit configured to provide, as the pre-shock conditioning electrostimulation, a series of electrostimulation pacing pulses to be delivered to the natural electrical conduction system of the heart, such as including to a location at or near a His bundle of the heart.

Example 6 can include, or can optionally be combined with the subject matter of one or any combination of Examples 1 through 5 to optionally include an interval between the electrostimulation pacing pulses that exceeds a refractory period of the Purkinje fibers of the heart.

Example 7 can include, or can optionally be combined with the subject matter of one or any combination of Examples 1 through 6 to optionally include a conditioning electrostimulation that comprises a post-shock conditioning electrostimulation, provided after the defibrillation shock, using the second electrical energy delivery circuit.

Example 8 can include, or can optionally be combined with the subject matter of one or any combination of Examples 1 through 7 to optionally include the second electrical energy delivery circuit configured to provide, as the post-shock conditioning electrostimulation, a series of electrostimulation pacing pulses to be delivered to the natural electrical conduction system of the heart, such as to a location at or near a His bundle of the heart.

Example 9 can include, or can optionally be combined with the subject matter of one or any combination of Examples 1 through 8 to optionally include a series of post-shock conditioning electrostimulation pacing pulses that comprises at least first, second, and third electrostimulation pacing pulses to be sequentially delivered to the natural electrical conduction system of the heart, such as to a location at or near a His bundle of the heart. Example 9 can optionally include or use an interval between the second and third adjacent electrostimulation pacing pulses that exceeds an interval between the first and second adjacent electrostimulation pacing pulses

Example 10 can include, or can optionally be combined with the subject matter of one or any combination of Examples 1 through 9 to optionally include a series of post-shock conditioning electrostimulation pacing pulses that are provided at a progressively slowing rate.

Example 11 can include, or can optionally be combined with the subject matter of one or any combination of Examples 1 through 10 to optionally include or use the processor circuit to initiate a defibrillation therapy in response to the identified fibrillation therapy. The defibrillation therapy of Example 11 can optionally include a pre-shock conditioning electrostimulation, provided using the second electrical energy delivery circuit, a defibrillation shock, provided after the pre-shock conditioning electrostimulation, using the first electrical energy delivery circuit, and a post-shock conditioning electrostimulation, provided after the defibrillation shock, using the second electrical energy delivery circuit.

Example 12 can include, or can optionally be combined with the subject matter of one or any combination of Examples 1 through 11 to optionally include the second electrical energy delivery circuit configured to provide, as the pre-shock conditioning electrostimulation, a first series of electrostimulation pacing pulses, to be delivered to the natural electrical conduction system of the heart, such as to a location at or near a His bundle of the heart, such as at a first electrostimulation rate. Example 12 can optionally include the second electrical energy delivery circuit configured to provide, as the post-shock conditioning electrostimulation, a second series of electrostimulation pacing pulses, to be delivered to the natural electrical conduction system of the heart, such as to a location at or near a His bundle of the heart, such as at a second electrostimulation rate. In Example 12, the first electrostimulation rate can optionally exceed the second electrostimulation rate.

Example 13 can include, or can optionally be combined with the subject matter of one or any combination of Examples 1 through 12 to optionally include the processor circuit configured to initiate a pre-shock conditioning electrostimulation in response to the identified defibrillation episode, provided using the second electrical energy delivery circuit. In Example 13, the processor circuit can be configured to identify a persistent fibrillation episode using the received signal representative of electrical activity of the heart, and in response to the identified persistent fibrillation episode, initiate the defibrillation shock provided using the first electrical energy delivery circuit.

Example 14 can include, or can optionally be combined with the subject matter of one or any combination of Examples 1 through 13 to optionally include the processor circuit configured to initiate the defibrillation therapy using a therapy parameter. Example 14 can include the processor circuit configured to identify whether the fibrillation episode persists, and when the fibrillation episode persists, update (e.g., automatically) the therapy parameter and initiate (e.g., automatically) the defibrillation therapy using the updated therapy parameter.

Example 15 can include, or can optionally be combined with the subject matter of one or any combination of Examples 1 through 14 to optionally include a therapy parameter that includes a waveform parameter. The waveform parameter can determine a characteristic of a waveform of the conditioning electrostimulation provided by the second electrical energy delivery circuit.

Example 16 can include, or can optionally be combined with the subject matter of one or any combination of Examples 1 through 15 to optionally include a therapy parameter that includes a timing parameter, such as used by the processor circuit, to provide a timing between the conditioning electrostimulation and the defibrillation shock.

Example 17 can include, or can optionally be combined with the subject matter of one or any combination of Examples 1-16 to include, subject matter (such as an apparatus, a method, a means for performing acts, or a machine readable medium including instructions that, when performed by the machine, that can cause the machine to perform acts), such as can include a detector circuit configured to receive a signal representative of electrical activity of a heart, a first electrical energy delivery circuit configured to provide a defibrillation shock to be delivered to the heart, a second electrical energy delivery circuit configured to provide a conditioning electrostimulation, in conjunction with the defibrillation shock, to be delivered to a location at or near a His bundle of the heart, and a processor circuit, coupled to the detector circuit and the first and second electrical energy delivery circuits. In Example 17, the processor circuit can optionally be configured to identify a ventricular fibrillation episode using the received signal representative of electrical activity of the heart, and, in response to the identified fibrillation episode, initiate a defibrillation therapy. In Example 17, the defibrillation therapy can optionally include a defibrillation shock provided using the first electrical energy delivery circuit, and a conditioning electrostimulation, provided using the second electrical energy delivery circuit. In Example 17, the conditioning electrostimulation can optionally include a pre-shock conditioning electrostimulation comprising a series of electrostimulation pacing pulses provided before the defibrillation shock at a first electrostimulation rate, and a post-shock conditioning electrostimulation comprising a series of electrostimulation pacing pulses provided after the defibrillation shock at a different second electrostimulation rate that is slower than the first electrostimulation rate.

Example 18 can include, or can optionally be combined with the subject matter of one or any combination of Examples 1-17 to include, subject matter (such as an apparatus, a method, a means for performing acts, or a machine readable medium including instructions that, when performed by the machine, that can cause the machine to perform acts), such as can include identifying automatically a fibrillation episode using an implantable medical device and a signal representative of electrical activity of a heart, and initiating a defibrillation therapy in response to the identified fibrillation episode. In Example 18, the defibrillation therapy can include providing a defibrillation shock using a first electrical energy delivery circuit of the implantable medical device, and providing a conditioning electrostimulation using a second electrical energy delivery circuit of the implantable medical device. In Example 18, the second electrical energy delivery circuit can be configured to deliver the conditioning electrostimulation to a natural electrical conduction system of the heart between the atrioventricular node and the Purkinje fibers, inclusive, such as at or near a His bundle of the heart.

Example 19 can include, or can optionally be combined with the subject matter of one or any combination of Examples 1 through 18 to optionally include automatically identifying the fibrillation episode, such as including distinguishing the fibrillation episode from a tachycardia episode.

Example 20 can include, or can optionally be combined with the subject matter of one or any combination of Examples 1 through 19 to optionally include providing the conditioning electrostimulation, including providing, using the second electrical energy delivery circuit, at least one of a pre-shock conditioning electrostimulation or a post-shock conditioning electrostimulation.

Each of these non-limiting examples can stand on its own, or can be combined in various permutations or combinations with one or more of the other examples.

The above detailed description includes references to the accompanying drawings, which form a part of the detailed description. The drawings show, by way of illustration, specific embodiments in which the invention can be practiced. These embodiments are also referred to herein as “examples.” Such examples can include elements in addition to those shown or described. However, the present inventors also contemplate examples in which only those elements shown or described are provided. Moreover, the present inventors also contemplate examples using any combination or permutation of those elements shown or described (or one or more aspects thereof), either with respect to a particular example (or one or more aspects thereof), or with respect to other examples (or one or more aspects thereof) shown or described herein.

In the event of inconsistent usages between this document and any documents so incorporated by reference, the usage in this document controls.

In this document, the terms “a” or “an” are used, as is common in patent documents, to include one or more than one, independent of any other instances or usages of “at least one” or “one or more.” In this document, the term “or” is used to refer to a nonexclusive or, such that “A or B” includes “A but not B,” “B but not A,” and “A and B,” unless otherwise indicated. In this document, the terms “including” and “in which” are used as the plain-English equivalents of the respective terms “comprising” and “wherein.” Also, in the following claims, the terms “including” and “comprising” are open-ended, that is, a system, device, article, composition, formulation, or process that includes elements in addition to those listed after such a term in a claim are still deemed to fall within the scope of that claim. Moreover, in the following claims, the terms “first,” “second,” and “third,” etc. are used merely as labels, and are not intended to impose numerical requirements on their objects.

Method examples described herein can be machine or computer-implemented at least in part. Some examples can include a computer-readable medium or machine-readable medium encoded with instructions operable to configure an electronic device to perform methods as described in the above examples. An implementation of such methods can include code, such as microcode, assembly language code, a higher-level language code, or the like. Such code can include computer readable instructions for performing various methods. The code may form portions of computer program products. Further, in an example, the code can be tangibly stored on one or more volatile, non-transitory, or non-volatile tangible computer-readable media, such as during execution or at other times. Examples of these tangible computer-readable media can include, but are not limited to, hard disks, removable magnetic disks, removable optical disks (e.g., compact disks and digital video disks), magnetic cassettes, memory cards or sticks, random access memories (RAMs), read only memories (ROMs), and the like.

The above description is intended to be illustrative, and not restrictive. For example, the above-described examples (or one or more aspects thereof) may be used in combination with each other. Other embodiments can be used, such as by one of ordinary skill in the art upon reviewing the above description. The Abstract is provided to comply with 37 C.F.R. §1.72(b), to allow the reader to quickly ascertain the nature of the technical disclosure. It is submitted with the understanding that it will not be used to interpret or limit the scope or meaning of the claims. Also, in the above Detailed Description, various features may be grouped together to streamline the disclosure. This should not be interpreted as intending that an unclaimed disclosed feature is essential to any claim. Rather, inventive subject matter may lie in less than all features of a particular disclosed embodiment. Thus, the following claims are hereby incorporated into the Detailed Description as examples or embodiments, with each claim standing on its own as a separate embodiment, and it is contemplated that such embodiments can be combined with each other in various combinations or permutations. The scope of the invention should be determined with reference to the appended claims, along with the full scope of equivalents to which such claims are entitled.

The claimed invention is:

1. An apparatus comprising:
a detector circuit configured to receive a signal representative of electrical activity of a heart;
a first electrical energy delivery circuit configured to provide a defibrillation shock to be delivered to the heart;
a second electrical energy delivery circuit configured to provide a conditioning electrostimulation, in conjunction with the defibrillation shock, to be delivered to a natural electrical conduction system of the heart between the atrioventricular node and the Purkinje fibers, inclusive; and
a processor circuit, coupled to the detector circuit and the first and second electrical energy delivery circuits, the processor circuit configured to:
identify a fibrillation episode using the received signal representative of electrical activity of the heart; and
in response to the identified fibrillation episode, initiate a defibrillation therapy comprising:
the defibrillation shock provided using the first electrical energy delivery circuit; and
the conditioning electrostimulation, provided using the second electrical energy delivery circuit.

2. The apparatus of claim 1, wherein the second electrical energy delivery circuit is configured to provide the conditioning electrostimulation, in conjunction with the defibrillation shock, to a location at or near a His bundle of the heart.

3. The apparatus of claim 1, wherein the processor circuit is configured to identify the fibrillation episode, including distinguishing the fibrillation episode from a tachycardia episode.

4. The apparatus of claim 1, wherein the processor circuit is configured to identify, as the fibrillation episode, a ventricular fibrillation episode that is contraindicated for an anti-tachycardia pacing therapy.

5. The apparatus of claim 1, wherein the conditioning electrostimulation comprises a pre-shock conditioning electrostimulation, provided before the defibrillation shock, using the second electrical energy delivery circuit.

6. The apparatus of claim 5, wherein the second electrical energy delivery circuit is configured to provide, as the pre-shock conditioning electrostimulation, a series of electrostimulation pacing pulses to be delivered to the natural electrical conduction system of the heart between the atrioventricular node and the Purkinje fibers, inclusive.

7. The apparatus of claim 1, wherein an interval between the electrostimulation pacing pulses exceeds a refractory period of the Purkinje fibers of the heart.

8. The apparatus of claim 1, wherein the conditioning electrostimulation comprises a post-shock conditioning electrostimulation, provided after the defibrillation shock, using the second electrical energy delivery circuit.

9. The apparatus of claim 8, wherein the second electrical energy delivery circuit is configured to provide, as the post-shock conditioning electrostimulation, a series of electrostimulation pacing pulses to be delivered to the natural electrical conduction system of the heart between the atrioventricular node and the Purkinje fibers, inclusive.

10. The apparatus of claim 9, wherein the series of post-shock conditioning electrostimulation pacing pulses comprises at least first, second, and third electrostimulation pacing pulses to be sequentially delivered to the natural electrical conduction system of the heart between the atrioventricular node and the Purkinje fibers, inclusive; and
wherein an interval between the second and third adjacent electrostimulation pacing pulses exceeds an interval between the first and second adjacent electrostimulation pacing pulses

11. The apparatus of claim 9, wherein the series of post-shock conditioning electrostimulation pacing pulses are provided at a progressively slowing rate.

12. The apparatus of claim 1, wherein in response to the identified fibrillation episode, the processor circuit is configured to initiate a defibrillation therapy comprising:
a pre-shock conditioning electrostimulation, provided using the second electrical energy delivery circuit;
the defibrillation shock, provided after the pre-shock conditioning electrostimulation, using the first electrical energy delivery circuit; and
a post-shock conditioning electrostimulation, provided after the defibrillation shock, using the second electrical energy delivery circuit.

13. The apparatus of claim 12, wherein the second electrical energy delivery circuit is configured to provide, as the pre-shock conditioning electrostimulation, a first series of electrostimulation pacing pulses, to be delivered to the natural electrical conduction system of the heart between the atrioventricular node and the Purkinje fibers, inclusive, at a first electrostimulation rate;
wherein the second electrical energy delivery circuit is configured to provide, as the post-shock conditioning electrostimulation, a second series of electrostimulation pacing pulses, to be delivered to the natural electrical conduction system of the heart between the atrioventricular node and the Purkinje fibers, inclusive, at a second electrostimulation rate; and
wherein the first electrostimulation rate exceeds the second electrostimulation rate.

14. The apparatus of claim 1, wherein the processor circuit is configured to:
in response to the identified defibrillation episode, initiate a pre-shock conditioning electrostimulation, provided using the second electrical energy delivery circuit;
identify a persistent fibrillation episode using the received signal representative of electrical activity of the heart; and
in response to the identified persistent fibrillation episode, initiate the defibrillation shock provided using the first electrical energy delivery circuit.

15. The apparatus of claim 1, wherein the processor circuit is configured to:
initiate the defibrillation therapy using a therapy parameter;
identify whether the fibrillation episode persists; and
when the fibrillation episode persists, update the therapy parameter and initiate the defibrillation therapy using the updated therapy parameter.

16. The apparatus of claim 15, wherein the therapy parameter includes at least one of:
a waveform parameter that determines a characteristic of a waveform of the conditioning electrostimulation provided by the second electrical energy delivery circuit; or
a timing parameter to provide a timing between the conditioning electrostimulation and the defibrillation shock.

17. An apparatus comprising:
a detector circuit configured to receive a signal representative of electrical activity of a heart;
a first electrical energy delivery circuit configured to provide a defibrillation shock to be delivered to the heart;
a second electrical energy delivery circuit configured to provide a conditioning electrostimulation, in conjunction with the defibrillation shock, to be delivered to a location at or near a His bundle of the heart; and
a processor circuit, coupled to the detector circuit and the first and second electrical energy delivery circuits, the processor circuit configured to:
identify a ventricular fibrillation episode using the received signal representative of electrical activity of the heart; and
in response to the identified fibrillation episode, initiate a defibrillation therapy comprising:
the defibrillation shock provided using the first electrical energy delivery circuit; and
the conditioning electrostimulation, provided using the second electrical energy delivery circuit, wherein the conditioning electrostimulation comprises:
a pre-shock conditioning electrostimulation comprising a series of electrostimulation pacing pulses provided before the defibrillation shock at a first electrostimulation rate; and
a post-shock conditioning electrostimulation comprising a series of electrostimulation pacing pulses provided after the defibrillation shock at a different second electrostimulation rate that is slower than the first electrostimulation rate.

18. A method comprising:
identifying automatically a fibrillation episode using an implantable medical device and a signal representative of electrical activity of a heart;
initiating a defibrillation therapy in response to the identified fibrillation episode, the defibrillation therapy comprising:
providing a defibrillation shock using a first electrical energy delivery circuit of the implantable medical device; and
providing a conditioning electrostimulation using a second electrical energy delivery circuit of the implantable medical device, wherein the second electrical energy delivery circuit is configured to deliver the conditioning electrostimulation to the natural electrical conduction system of the heart between the atrioventricular node and the Purkinje fibers, inclusive.

19. The method of claim 18, wherein the identifying automatically the fibrillation episode includes distinguishing the fibrillation episode from a tachycardia episode.

20. The method of claim 18, wherein the providing the conditioning electrostimulation comprises providing, using the second electrical energy delivery circuit, at least one of a pre-shock conditioning electrostimulation or a post-shock conditioning electrostimulation.
